(19) United States
(12) Patent Application Publication
(10) Pub. No.: US 2022/0387545 A1
(43) Pub. Date: Dec. 8, 2022

(54) COLLAGEN HYDROLYSATE AS ACTIVE SUBSTANCE AGAINST PERIODONTITIS OR GINGIVITIS

(71) Applicant: GELITA AG, Eberbach (DE)

(72) Inventors: Stephan HAUSMANNS, Heidelberg (DE); Hans-Ulrich FRECH, Weinheim (DE); Steffen OESSER, Glücksburg (DE); Tonja LIPP, Princeton, NJ (US)

(21) Appl. No.: 17/889,754

(22) Filed: Aug. 17, 2022

Related U.S. Application Data
(63) Continuation of application No. PCT/EP2020/086865, filed on Dec. 17, 2020.

(30) Foreign Application Priority Data
Feb. 18, 2020 (DE) 102020104279.5

(51) Int. Cl. A61K 38/01; A61P 1/02

(52) U.S. Cl. CPC A61K 38/014; A61P 1/02

(57) ABSTRACT

The following invention relates to collagen hydrolysate for use as an active substance in the treatment and/or prophylaxis of periodontitis or gingivitis.

### FIELD OF THE INVENTION

Periodontitis is an inflammation of the so-called periodontal apparatus, i.e. the entirety of the tissue holding a tooth in the jaw bone is affected by this disease. Colloquially, although medically incorrect, this disease is often also referred to as periodontosis.

By contrast, gingivitis is merely an inflammation of the gum (gingiva) or the gumline. Gingivitis, however, is often a precursor stage to periodontitis and may develop into the latter if left untreated. Within the scope of the present description, where reference is made to a treatment or prophylaxis of periodontitis, this therefore also always includes a treatment or prophylaxis of gingivitis, unless the context under consideration indicates otherwise.

In developed countries, periodontitis and gingivitis are among the most frequently occurring diseases. In Germany, it currently affects approximately 23 million people.

Periodontitis can be acute, but very often takes a chronic course. Since it is usually painless in the initial stage, it is often identified too late, frequently in patients aged from 40 to 50 years. Typical symptoms are redness, swelling, bleeding gums and gum recession. The latter leads to an increased pain sensitivity of the exposed tooth neck. In a further progressed stage, the periodontitis may lead to a loosening of the teeth and ultimately to tooth loss.

In addition to the negative and partly serious effects on the periodontal apparatus or the teeth, however, the periodontitis may also have systemic effects. On the one hand, the inflamed periodontal apparatus facilitates the infiltration of pathogens into the bloodstream, and on the other hand proinflammatory messenger substances are produced by the affected tissue. Chronic periodontitis thus leads in some instances to a significantly increased risk for cardiovascular diseases, respiratory illnesses, heart attacks and strokes.

A causal treatment of periodontitis has not been known before now. The progression of the disease, however, can be contained, in particular by a regular mechanical removal of supra- and subgingival plaque, although surgical incision into the gum may also be necessary at an advanced stage. In some cases, the administration of antibiotics is additionally expedient. On the whole, this treatment of periodontitis is protracted and costly.

### BRIEF SUMMARY OF THE INVENTION

Against this background, the object of the invention is to propose an active substance for the treatment and/or prophylaxis of periodontitis or gingivitis.

The present invention relates to collagen hydrolysate for use as an active substance in the treatment and/or prophylaxis of periodontitis or gingivitis.

### BRIEF DESCRIPTION OF THE SEVERAL VIEWS OF THE DRAWING(S)

The Figs show, specifically:

FIG. 1: stimulation of the synthesis of matrix proteins by a bovine collagen hydrolysate of approximately 2,000 Da;

FIG. 2: stimulation of the synthesis of matrix proteins by a porcine collagen hydrolysate of approximately 3,000 Da; and

FIG. 3: stimulation of the synthesis of matrix proteins by a bovine collagen hydrolysate of approximately 6,000 Da.

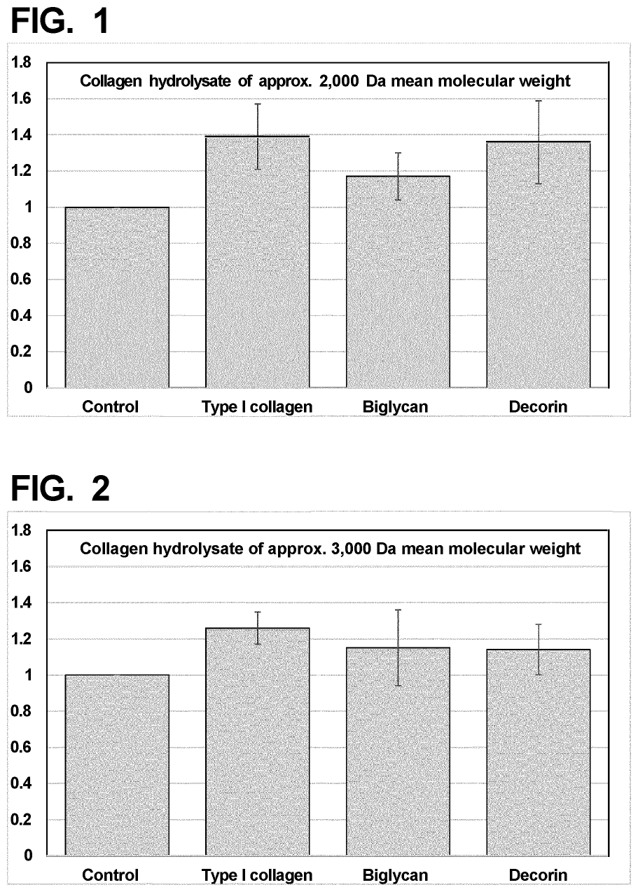

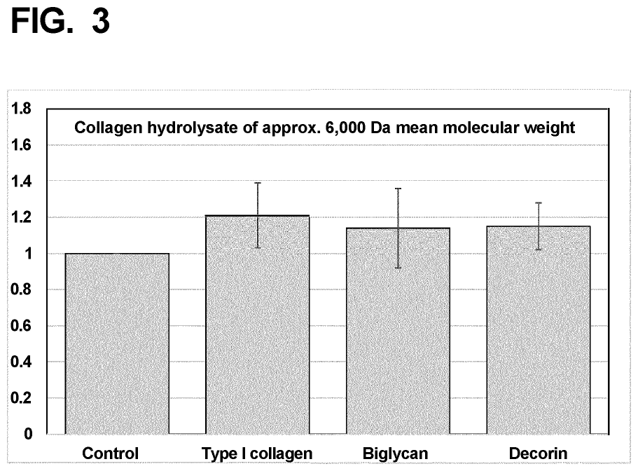

### DETAILED DESCRIPTION OF THE INVENTION

To achieve this object, the use of collagen hydrolysate as an appropriate active substance is proposed in accordance with the present invention. It has been possible to demonstrate clearly the efficacy of collagen hydrolysate in the treatment of periodontitis on the basis of a double-blind, placebo-controlled clinical study, the results of which are presented in detail further below.

Physiological effects of collagen hydrolysate have already been known for a relatively long time, in particular in conjunction with osteoporosis or in joint complaints. Positive effects of collagen hydrolysate on the health of the skin have also been described already, for example in international patent application WO 2012/065782 A2. Nevertheless, the high efficacy of collagen hydrolysate in the treatment of periodontitis is surprising.

Collagen hydrolysate, as a by-product of animal starting materials which are also used as foodstuffs, is a completely harmless product from a health point of view with no known harmful side effects. It does not require legal approval as a medicament, and can be marketed and used in particular in the form of a dietary supplement. Use of collagen hydrolysate as a dietary supplement, as a prescription-free (OTC) medicament or as a prescription medicament (in particular in combination with other active substances) is included within the scope of the present invention. Regardless of this classification, collagen hydrolysate is an active therapeutic sub stance.

Collagen hydrolysate ideally is administered orally. It is known that the peptides of collagen hydrolysate are resorbed in the intestine at least to a certain extent even with relatively high molecular weights of up to 10,000 Da.

The specific dosage form of collagen hydrolysate can be constituted by a powder, a solution, a tablet or a capsule.

Further preferred dosage forms are chewing gums, in particular based on compressed compositions, sucking lozenges, or similar products which remain in the mouth for a relatively long time, and drinkable mouth rinses. This type of administration, besides the systemic effect, also allows for an intense local effect of the collagen hydrolysate on the gum.

The daily dose of the administered collagen hydrolysate, in particular in the case of oral administration, is favourably from approximately 1 to approximately 20 g, preferably from approximately 2 to approximately 15 g, more preferably from approximately 3 to approximately 10 g. In the clinical study it was possible to demonstrate efficacy with a daily dose of 5 g collagen hydrolysate.

A preferred embodiment of the invention relates to the treatment of periodontitis or gingivitis, i.e. the administration of collagen hydrolysate to a patient with a corresponding disease, in particular with chronic periodontitis. In this case the administration is performed favourably, in particular in the case of an advanced disease, in combination with a further therapy for periodontitis. This further therapy comprises in particular the mechanical removal of supra- and subgingival plaque (biofilm and tartar). Although the administration in accordance with the invention of collagen hydrolysate is no replacement for the removal of plaque, it can positively influence the progression of the disease and can significantly reduce the damage caused by periodontitis. In some circumstances the severity of the disease, which ultimately also necessitates surgical measures, can be prevented by the administration of collagen hydrolysate.

A further embodiment of the invention relates to the prophylaxis of periodontitis or gingivitis. In this regard, the administration of collagen hydrolysate to a patient in whom one or more risk factors for these diseases are present is particularly advantageous. These risk factors for periodontitis include, in particular, a weakened immune system, being a smoker, hormonal changes (for example menopause) and certain genetic disorders (for example Down's syndrome). Existing gingivitis can be considered generally to be a risk factor for periodontitis, and therefore treatment of gingivitis by administering collagen hydrolysate simultaneously constitutes a prophylaxis for periodontitis.

The collagen hydrolysate as an active substance in accordance with the invention typically has a mean molecular weight of from 500 to 15,000 Da, preferably from 1,000 to 8,000 Da, more preferably from 1,500 to 5,000 Da, most preferably from 1,800 to 2,200 Da. In these statements the weight-average molecular weight is always meant, which can be determined in particular by gel permeation chromatography.

The collagen hydrolysate is preferably produced by enzymatic hydrolysis of a collagen-containing starting material. In particular, endopeptidases or exopeptidases of microbial or plant origin are used for this hydrolysis. Collagen hydrolysates in the desired molecular weight range can be produced by suitable selection of the peptidases and the hydrolysis conditions.

The collagen-containing starting material is generally selected from skin or bone of vertebrates, preferably of mammals or birds, and in particular from the skin of cattle or pigs (bovine split hide or pork rind respectively). Alternatively, the collagen-containing starting material can be selected from skin, bone and/or scales of fish, in particular cold-water fish or tropical fish.

The collagen hydrolysate can be produced either in a one-stage method from these starting materials or by means of the intermediate stage gelatine; in the latter case, gelatine both of type A and of type B can be used.

The collagen hydrolysate is preferably produced by the successive action of at least two endoproteases having a different specificity, in particular of at least two different metalloproteases and/or serine proteases, i.e. of proteases that cleave the amino acid sequence of the collagen molecules before and/or after specific amino acids. The metalloproteases and/or serine proteases are expediently enzymes from the microorganisms Bacillus subtilis, Bacillus licheniformis, Bacillus amyloliquefaciens, Aspergillus oryzae and Aspergillus melleus.

Due to the selection of suitable endoproteases, not only can a specific molecular weight distribution of the collagen hydrolysate be obtained, but the type of amino acids at the termini of the peptides contained in the hydrolysate is also influenced. In this respect it is preferred, for example, if at least 50% of the N-terminal amino acids of the collagen hydrolysate are hydrophobic amino acids, in particular alanine, leucine and isoleucine.

Alternatively to the enzymatic hydrolysis, the collagen hydrolysate can be produced by recombinant gene expression within the scope of the invention. By use of natural collagen sequences, in particular from cattle or pigs, and expression thereof in genetically modified cells (for example yeasts, bacteria or plant cells, in particular tobacco), products can be produced that are substantially identical to the hydrolysis products of the corresponding collagen-containing raw materials. It is possible here to obtain a narrower or precisely specified molecular weight distribution.

The present invention also relates to a therapeutic method for the treatment and/or prophylaxis of periodontitis or gingivitis comprising the administration of collagen hydrolysate to a patient. Advantages and preferred embodiments of this method have already been described in conjunction with the collagen hydrolysate in accordance with the invention.

The efficacy of collagen hydrolysate in the treatment of periodontitis will be explained in greater detail on the basis of the clinical study described hereinafter and on the basis of cellular tests performed on gingival fibroblasts.

### EXAMPLE 1

### 1. Clinical Study

### 1.1 Study Design

The double-blind, placebo-controlled clinical study was performed at the Department for Periodontology of the University Hospital Würzburg with 43 patients who, due to existing periodontitis, attended there for regular treatment (removal of subgingival plaque twice to four times per year). All study participants had at least three teeth with a gingival index of 1 or 2 (see below) and a probing depth of the gingival pockets of at least 3 mm. The participants were aged between 35 and 70 years and had a BMI between 24 and 30.

The participants were divided randomly into a treatment group with 23 patients and a placebo group with 20 patients, with neither the participants nor the medical staff being informed of the division of the individual participants.

The participants in the treatment group received a daily dose of 5 g collagen hydrolysate during the entire study period of 90±14 days. A collagen hydrolysate produced by enzymatic hydrolysis of bovine collagen and having a mean molecular weight of approximately 2,000 Da was used, distributed by the applicant GELITA AG under the name VERISOL B. The production of VERISOL B corresponds substantially to the production method described in WO 2012/065782 A2.

The participants in the placebo group, instead of the collagen hydrolysate, received a corresponding daily dose of a placebo, which was indistinguishable from the collagen hydrolysate in respect of its packaging, texture and taste.

At the start of the study (V1 on day 0), all study participants were given a thorough examination, and a series of parameters and indices were determined, characterising the status of the periodontitis. The patients then underwent a professional tooth cleaning process with mechanical removal of supra- and subgingival plaque.

The same parameters and indices were determined again with a second examination (V2 after 60±14 days) and with a third examination at the end of the study period (V3 after 90±14 days). Not all examinations could be performed for all original 43 study participants; the number of examined participants from each of the two groups is stated in the following evaluation.

A comparison of the changes to the determined parameters and indices between the treatment group and the placebo group makes it possible to determine the efficacy of the collagen hydrolysate in the treatment of periodontitis.

### 1.2 Bleeding on Probing

For the bleeding on probing (BoP) test, a periodontal probe is introduced into the gingival pockets. Any bleeding that occurs here is a sure sign of acute inflammation.

The bleeding on probing test was performed at examinations V1, V2 and V3, in each case using a periodontal probe PCP-11; the percentage of all gingival pockets for which bleeding occurred was detected here as BoP [%] for each patient. The results are shown in Table 1 below.

TABLE 1
Bleeding on probing (BoP)

| | | | | | p-value |
|---|---|---|---|---|---|
| | | | BoP [%] | | (Mann- |
| | | | Mean | | Whitney |
| Examination | Group | Number | value | SD | U test) |
| V1 | all participants | 41 | 13.1 | 11.0 | 0.465 |
| | treatment group | 21 | 12.3 | 11.9 | |
| | placebo group | 20 | 14.0 | 10.2 | |
| V2 | all participants | 39 | 7.83 | 9.75 | 0.047 |
| | treatment group | 20 | 4.93 | 6.95 | |
| | placebo group | 19 | 10.9 | 11.4 | |
| V3 | all participants | 38 | 5.75 | 7.59 | 0.033 |
| | treatment group | 20 | 3.52 | 5.77 | |
| | placebo group | 18 | 8.24 | 8.69 | |

These results show that the percentage of bleeding gingival pockets in all patients during the study period decreased, but significantly more so for the participants in the treatment group than for the participants in the placebo group.

### 1.3 Plaque Control Record

To determine the plaque control record (PCR) according to O'Leary et al. 1972, the plaque was dyed and its extent along the entire gumline (i.e. on all sides of the tooth) was optically recorded. The parameter is formed from the number of plaque-positive surfaces in relation to the total number of assessed surfaces in percent.

The results for the PCR [%] in examinations V1, V2 and V3 are shown in Table 2 below.

TABLE 2
Plaque control record (PCR)

| | | | | | p-value |
|---|---|---|---|---|---|
| | | | PCR [%] | | (Mann- |
| | | | Mean | | Whitney |
| Examination | Group | Number | value | SD | U test) |
| V1 | all participants | 43 | 26.0 | 18.4 | 0.408 |
| | treatment group | 23 | 24.4 | 19.5 | |
| | placebo group | 20 | 27.8 | 17.3 | |
| V2 | all participants | 39 | 16.7 | 13.3 | 0.054 |
| | treatment group | 20 | 12.7 | 11.3 | |
| | placebo group | 19 | 20.9 | 14.1 | |
| V3 | all participants | 38 | 12.0 | 12.9 | 0.098 |
| | treatment group | 20 | 7.59 | 8.17 | |
| | placebo group | 18 | 16.9 | 15.5 | |

The results show that the PCR in all patients during the study period decreased, but significantly more so for the participants in the treatment group than for the participants in the placebo group.

### 1.4 Gingival Index

The gingival index (GI) according to Löe 1967 (modified according to Lobene et al. 1986) is given from the visual assessment of the state of acute inflammation of the gum on the buccal side in accordance with the following schema:

Level 0: no inflammation

Level 1: colour change to an area of gum, but not the entire gumline or gingival papilla

Level 2: colour change incorporating the entire gumline or gingival papilla

Level 3: as for level 2, but pronounced redness

For each patient the gingival index GI is given as the mean value of the indices of all teeth. The examination results for the gingival index are shown in Table 3 below.

TABLE 3
Gingival index (GI)

| | | | | | p-value |
|---|---|---|---|---|---|
| | | | GI [%] | | (Mann- |
| | | | Mean | | Whitney |
| Examination | Group | Number | value | SD | U test) |
| V1 | all participants | 43 | 0.465 | 0.248 | 0.073 |
| | treatment group | 23 | 0.532 | 0.275 | |
| | placebo group | 20 | 0.387 | 0.190 | |
| V2 | all participants | 39 | 0.231 | 0.164 | 0.003 |
| | treatment group | 20 | 0.153 | 0.109 | |
| | placebo group | 19 | 0.313 | 0.175 | |
| | all participants | 38 | 0.182 | 0.181 | |
| V3 | all participants | 38 | 0.182 | 0.181 | 0.035 |
| | treatment group | 20 | 0.130 | 0.158 | |
| | placebo group | 18 | 0.240 | 0.191 | |

The results show that the gingival index in all patients during the study period reduced, i.e. the acute extent of the gum inflammation decreased. However, this decrease is significantly greater for the participants in the treatment group than for the participants in the placebo group.

### 1.5 PISA Score

The PISA score indicates the extent of the area of periodontal inflammation (periodontal inflamed surface area) in mm². The PISA score can be calculated from the above-described parameters; the results are shown in Table 4 below.

TABLE 4
PISA score

| | | | | | p-value |
|---|---|---|---|---|---|
| | | | PISA [mm²] | | (Mann- |
| | | | Mean | | Whitney |
| Examination | Group | Number | value | SD | U test) |
| V1 | all participants | 41 | 212.1 | 205.1 | 0.481 |
| | treatment group | 21 | 206.0 | 242.4 | |
| | placebo group | 20 | 218.4 | 163.0 | |
| V2 | all participants | 39 | 147.8 | 201.9 | 0.040 |
| | treatment group | 20 | 91.6 | 151.9 | |
| | placebo group | 19 | 206.9 | 233.4 | |
| V3 | all participants | 38 | 101.0 | 150.0 | 0.024 |
| | treatment group | 20 | 58.7 | 115.4 | |
| | placebo group | 18 | 148.0 | 172.2 | |

The results show a significant reduction in the PISA scores for all patients during the study period, but for this parameter as well the change for the participants in the treatment group is much more pronounced than for the participants in the placebo group.

### 1.6 Summary

The clinical study shows, on the basis of the parameters of bleeding on probing, plaque control record, gingival index and PISA score, that a significantly greater improvement in periodontitis was achieved by the daily administration of 5 g collagen hydrolysate than in the placebo group. This result was achieved over a relatively short period of time of 3 months, with both groups receiving the same conventional treatment (supra- and subgingival removal of plaque).

The results thus clearly demonstrate the efficacy of collagen hydrolysate as an active substance in the treatment and/or prophylaxis of periodontitis or gingivitis.

### 2. Cellular Tests on Gingival Fibroblasts

The gum consists of gingival fibroblasts (gum cells) and the extracellular matrix which is formed by these cells and contains different types of collagen and proteoglycans. In the case of periodontitis or gingivitis, the balance between the biosynthesis and the degradation of these matrix proteins is typically also disturbed. A stimulation of the synthesis of matrix proteins by the fibroblasts may thus counteract periodontitis or gingivitis.

In the cellular tests described hereinafter, the stimulating effect of collagen hydrolysate with different mean molecular weights on the synthesis of the matrix proteins type I collagen, biglycan and decorin in human gingival fibroblasts was examined in vitro. This was achieved by determining the expression of the corresponding mRNA by means of real-time PCR and a semi-quantitative evaluation (based on a control without collagen hydrolysate).

The human gingival fibroblasts were isolated by enzymatic digestion from surgically removed gum tissue and were cultured in HAMs F12 medium, which was supplemented with 10% foetal calf serum, 20 U/ml penicillin streptomycin, and 50 μg/ml partricin. Once confluence of 80% had been reached, the culture medium was replaced by fresh medium supplemented with 0.5 mg/ml collagen hydrolysate.

The cellular tests were performed with three different collagen hydrolysates: (1) a bovine collagen hydrolysate with a mean molecular weight of approximately 2,000 Da, which was also used in the above clinical study, (2) a porcine collagen hydrolysate with a mean molecular weight of approximately 3,000 Da, and (3) a bovine collagen hydrolysate with a mean molecular weight of approximately 6,000 Da.

Following incubation of the fibroblasts for 24 h in the presence of the particular collagen hydrolysate (or without collagen hydrolysate in the case of the control), the cells were harvested and lysed, then the total RNA was extracted and precipitated.

The results of the semi-quantitative evaluation of the amounts of mRNA for type I collagen, biglycan and decorin are shown in the Figs.

All three examined collagen hydrolysates therefore lead to a significant stimulation of the synthesis of the extracellular matrix proteins constituted by type I collagen, biglycan and decorin by human gingival fibroblasts in vitro. This effect is strongest for the collagen hydrolysate with a mean molecular weight of approximately 2,000 Da, especially with regard to the stimulation of the synthesis of type I collagen and decorin.

This result confirms the efficacy of collagen hydrolysate in the treatment and/or prophylaxis of periodontitis or gingivitis. At least one of the mechanisms of action here is the strengthening of the extracellular matrix of the gum by an intensified synthesis of matrix proteins under the action of collagen hydrolysate.

1. A method for the treatment and/or prophylaxis of periodontitis or gingivitis, the method comprising administering collagen hydrolysate to a patient.

2. The method in accordance with claim 1, wherein the collagen hydrolysate is in the form of a dietary supplement or a medicament.

3. The method in accordance with claim 1, wherein the collagen hydrolysate is administered orally.

4. The method in accordance with claim 3, wherein the collagen hydrolysate is administered in the form of a powder, a solution, a tablet or a capsule.

5. The method in accordance with claim 1, wherein the collagen hydrolysate is administered in a daily dose of from approximately 1 to approximately 20 g.

6. The method in accordance with claim 1, wherein the patient has periodontitis or gingivitis.

7. The method in accordance with claim 6, wherein the collagen hydrolysate is administered in combination with a further therapy for periodontitis or gingivitis comprising a mechanical removal of supra- and subgingival plaque.

8. The method in accordance with claim 1, wherein the patient has one or more risk factors for periodontitis or gingivitis.

9. The method in accordance with claim 1, wherein the collagen hydrolysate has a mean molecular weight of from 500 to 15,000 Da.

10. The method in accordance with claim 1, wherein the collagen hydrolysate is obtained by enzymatic hydrolysis of a collagen-containing starting material.

11. The method in accordance with claim 10, wherein the collagen-containing starting material is selected from skin or bone of vertebrates.

12. The method in accordance with claim 10, wherein the collagen hydrolysate is obtained by the successive action of at least two endoproteases having a different specificity, wherein the at least two endoproteases comprise at least two different metalloproteases and/or serine proteases.

13. The method in accordance with claim 12, wherein the metalloproteases and/or serine proteases are selected from enzymes from the microorganisms Bacillus subtilis, Bacillus licheniformis, Bacillus amyloliquefaciens, Aspergillus oryzae and Aspergillus melleus.

14. The method in accordance with claim 12, wherein at least 50% of the N-terminal amino acids of the collagen hydrolysate are hydrophobic amino acids selected from alanine, leucine and isoleucine.

15. The method in accordance with claim 1, wherein the collagen hydrolysate is produced by recombinant gene expression.

16. The method in accordance with claim 5, wherein the collagen hydrolysate is administered in a daily dose of from approximately 2 to approximately 15 g.

17. The method in accordance with claim 16, wherein the collagen hydrolysate is administered in a daily dose of from approximately 3 to approximately 10 g.

18. The method in accordance with claim 9, wherein the collagen hydrolysate has a mean molecular weight of from 1,000 to 8,000 Da.

19. The method in accordance with claim 18, wherein the collagen hydrolysate has a mean molecular weight of from 1,500 to 5,000 Da.

20. The method in accordance with claim 19, wherein the collagen hydrolysate has a mean molecular weight of from 1,800 to 2,200 Da.
